(19) United States
(12) Patent Application Publication
(10) Pub. No.: US 2024/0245768 A1
(43) Pub. Date: Jul. 25, 2024

(54) Polyclonal Antibodies to Treat Respiratory Syncytial Virus

(71) Applicant: LACTIGA, INC., Toronto (CA)

(72) Inventors: Viraj Mane, Highland Park, NJ (US); Rikin Mehta, Chester, NJ (US)

(73) Assignee: LACTIGA, INC., Toronto, ON (CA)

(21) Appl. No.: 18/562,606

(22) PCT Filed: May 20, 2022
(86) PCT No.: PCT/IB2022/054763

§ 371 Date: Nov. 20, 2023

Related U.S. Application Data
(60) Provisional application No. 63/190,893, filed on May 20, 2021.

(51) Int. Cl. A61K 39/395; A61K 9/00; A61K 35/20; A61P 31/14

(52) U.S. Cl. CPC A61K 39/39508; A61K 9/0078; A61K 35/20; A61P 31/14

(57) ABSTRACT

A composition and method of preparing the composition is provided. The composition is for inhalation to treat respiratory syncytial virus. The composition includes a poly clonal antibody derived from human breastmilk. In particular, the composition includes intact antibodies representing more than about 81% of total antibodies. The total antibodies include intact antibodies and degraded antibodies.

### BACKGROUND

Respiratory syncytial virus (RSV) is one of the most common respiratory viruses. It generally causes cold-like symptoms especially in young children. For many, the symptoms are minor and generally considered more of a nuisance; most people affected by RSV recover within a couple of weeks. However, for other individuals including those who are very young, very old, and/or immunocompromised, RSV can be serious and result in bronchiolitis and pneumonia that lead to over 100,000 hospitalizations and 10,000 deaths per year in the US according to CDC reports from December 2020. RSV recurs every winter, is highly contagious, and has no approved vaccine, underscoring its negative impact on public health.

To treat serious cases of RSV infections, commercial monoclonal antibody therapeutics have been developed. As an example, Palivizumab, sold under the brand name SYNAGIS, is a monoclonal antibody produced by recombinant DNA technology used to prevent severe disease caused by RSV infections. However, there are significant shortcomings to the recombinant monoclonal antibody approach, as described below, with respect to biology/immunology, cost, and patient adherence:

Monoclonal antibodies recognize only one antigen associated with one pathogenic strain and may not retain efficacy against emerging variants with different surface characteristics.

Ramping up recombinant antibody production facilities to deliver therapeutics at sufficient scale for public health purposes is cost-prohibitive and time-intensive.

Taking SYNAGIS as an example of monoclonal antibody administration, in high-risk children it is dosed at 15 mg/kg intramuscularly into the thigh every month throughout the RSV season which in the northern hemisphere typically lasts from November through April, making it an expensive prophylactic measure that can require repetitive hospital or clinic visits.

To address the shortcomings of existing monoclonal antibody therapeutics, a novel polyclonal antibody therapeutic is described herein.

### BRIEF DESCRIPTION OF THE DRAWINGS

Reference will now be made, by way of example only, to the accompanying drawings in which:

FIG. 1 are microscopy image results from human HEp-2 cells in the presence or absence of GFP-tagged RSV that was preincubated in the presence or absence of human milk-derived polyclonal antibodies, where these antibodies are referred to herein as LCTG-001; and

FIG. 2 are cellular infection averages and standard deviations calculated from GFP quantification of the in vitro experiment described in FIG. 1 to test the effectiveness of LCTG-001, frozen in storage for one year, for neutralizing RSV in HEp-2 human airway cells; and

FIG. 3 are cellular infection averages and standard deviations from an in vitro experiment to test the effectiveness of LCTG-001, frozen in storage for three years, for neutralizing RSV in HEp-2 human airway cells.

### DETAILED DESCRIPTION

Treatments for respiratory syncytial virus (RSV) infections are known. For example, monoclonal antibodies, such as SYNAGIS (a humanized IgG1 monoclonal antibody), may be produced using recombinant DNA technology. Although commercial monoclonal antibodies may be successful in treating RSV infections, they present some drawbacks. For example, monoclonal antibodies are generally engineered to recognize a specific surface feature of one pathogenic strain and may not retain efficacy against emerging variants with different surface characteristics, as has been observed with antibody therapeutics against the highly-mutating influenza virus and the novel coronavirus, SARS-CoV-2.

As another example, scale-up of recombinant antibody production, one of the most common forms of producing RSV therapeutics, is too cost-prohibitive and time-intensive to be used for general public health purposes. In addition, for some therapeutics, the delivery method may be in the form of a periodic intramuscular injection during a period of high RSV activity, generally lasting from November to April in North America, creating a requirement for hospital visits despite the risk of nosocomial infections associated with this season. Furthermore, it can be appreciated that intramuscular injections are not associated with efficient delivery of a therapeutic to the airways or lungs.

RSV circulates widely among the human population and frequently re-infects individuals. Consequently, it is widely accepted that most people have been previously exposed to RSV from infancy onward. Accordingly, defining the mechanisms that do (or do not) lead to durable and effective immunity against RSV infection is difficult and not very well understood. Furthermore, it has been suggested that specific antibodies to RSV are not a primary component of the immune response to RSV infection, at least in the murine model.

A polyclonal antibody therapeutic is provided and positioned to replace monoclonal recombinant antibody therapeutics to treat RSV. In particular, polyclonal antibodies are to be derived from human milk antibodies. Human milk antibodies are naturally polyclonal, meaning they can bind to multiple features on a single pathogen such as RSV. This provides greater coverage of antigens and increases the likelihood of retaining efficacy against both existing and emerging variants, unlike recombinant monoclonal antibodies which can only bind one specific antigenic epitope. In the present polyclonal antibody therapeutic, the antibodies are to be inhaled and accumulated in the respiratory tract which is the primary affected tissue with respect to the replication and accumulation of RSV. The manner by which the antibodies are delivered via inhalation is not limited and may include the use of an inhaler device, a nebulizer device, or other methods. This method of delivery may result in the greater accumulation of antibodies to neutralize the RSV at the primary site of infection, when compared to monoclonal antibodies that are injected or infused into the blood and therefore do not accumulate to a similar extent within the respiratory tract.

It is to be understood by a person skilled in the art with the benefit of this description that human milk antibodies are primarily (around 90%) of the dimeric IgA isotype. The dimeric IgA structure is distinct from recombinant monoclonal antibodies that are typically of the monomeric IgG isotype (e.g. SYNAGIS). Dimeric IgA, unlike IgG, is known to have a favorable stability profile in the respiratory tract, which further provides advantages for an inhaled delivery method instead of the injection or infusion methods typically used for biologics. Various properties of breastmilk (including but not limited to viscosity, antibody composition, antibody concentration, and antibody titer) differ from serum or hybridoma supernatant, therefore processes specific to maternal antibody capture have been previously unknown in the art, and are distinct from existing methods of (non-maternal) antibody capture. In the present example, various steps are described that, when combined, are used to extract, purify, and stabilize maternal antibodies from breastmilk.

In an example, immunoglobulin extraction from milk involves a milk clarification step, and use of 65% ammonium sulfate for precipitation, both of which are distinct from known serum or hybridoma-associated techniques.

The preferred examples of the formulations described herein may provide for long-term storage of antibodies at room temperature which is an ongoing challenge in the development of antibody preparations. In addition, the formulations provide for longer storage when stored at temperatures below room temperature, such as at about −20° C., as described in greater detail below.

The formulations and processes of the examples described herein provide improved stability for the IgA subtype antibody, for example, for packaging this antibody to provide beneficial thermostability and easy handling, to improve delivery to a patient via inhalation.

The processes described herein combine biochemical and analytical techniques to extract maternal antibodies from the breastmilk of mammals, including humans. While the formulations and processes described herein are specific to human antibodies formulated for human consumption, the concept may be applied to other mammals that produce milk. For example, the formulations and processes described herein may be provided to deliver cow antibodies formulated for cows or sheep antibodies formulated for sheep to provide antibodies for inhalation to treat respiratory diseases. Furthermore, as a human recipient may benefit from the inhalation of human antibodies to treat RSV, there may be a benefit to inhaling antibodies from a different species. For example, some bovine immunoglobulin products have been designed for human use, as in the serum-derived bovine immunoglobulin used to manage enteropathy in human patients in people whose ability to digest is impaired.

In an example, the process of extraction of maternal antibodies begins with the collection of breastmilk from a mammalian donor. Breastmilk may be expressed from the breast by using physical stimulation, a mechanical pump apparatus, a combination of these, or any other method that stimulates the flow of breastmilk from the milk duct. In this example, the process may involve collecting the sample, transporting the sample via refrigeration, and processing the milk into the formulation for inhalation soon after collection. In situations where sample processing cannot occur rapidly, such as within about 24 hours, samples may be refrigerated during transport, then frozen until ready for processing. Alternatively, the sample may also be directly frozen after acquisition. It is to be appreciated by a person of skill with the benefit of this description that a goal may be to reduce the time that whole breastmilk sits at or near ambient room temperature. In addition, freeze/thaw cycles of the breastmilk are also to be reduced as these may damage the structure of the antibodies in the breastmilk.

To promote the sterile collection of breastmilk, the breast and nipple will be treated with an alcohol wipe or other cleanser. Cleaners, including alcohol wipes or soaps, that are already in use to sterilize/disinfect human skin surfaces are suitable for this purpose. However, strong acids and bases should be avoided as they may affect the pH and/or acidity of the collected breastmilk sample prior to sample processing. Similarly, the pump apparatus used to promote the flow of breastmilk should be wiped with cleanser or sterilized with common disinfection techniques before being applied to the breast.

In this example, breastmilk may be collected into suitable bags or containers. The bags or containers may be made of materials such as plastic, metal, polymer, or combinations thereof, and may be selected based on tolerance to various temperatures and lack of cross-reactivity with antibodies. For example, commercial breastmilk storage bags are typically flexible for easy storage, have double zipper seals to prevent leakage, and are stable at various temperatures including room temperature (about 25° C.), refrigeration (about 4° C.), and freezing temperature (below or about −18° C.).

To increase the stability of antibodies and reduce antibody degradation, storage of breastmilk beyond a few days should be carried out at the freezing temperature. However, storage of breastmilk under other conditions may still be compatible with the antibody extraction method, especially if the storage time spent under refrigeration or room temperature is limited.

The pasteurization heat treatment process is often carried out as part of a typical milk processing protocol. However, pasteurization may denature and degrade antibodies and is therefore not carried out since pasteurization may be incompatible with the goal of maximizing antibody preservation and stabilization.

Antibody extraction or purification methods may range from crude (nonspecific) to highly specific. As used herein, “crude” refers to a method that does not distinguish among antibody subtypes, and retains multiple (or all) antibody subtypes; while “specific” can refer to class-specific or antigen-specific affinity, as described below.

In an example, the goals of the extraction step are to capture the component of interest, such as antibodies, and preferably all breastmilk antibody subtypes; wash away all other unwanted components, such as water, fats, sugars, proteins, small molecules, and any pathogens or other environmental compounds that may be contaminating the sample; and elute the purified antibody fraction. The method of the present example is designed to capture the broadest possible spectrum of antibodies. Therefore, the preference is for nonspecific methods that facilitate maximum antibody capture. Preferably, all antibodies may be collected from each breastmilk sample to increase protection against pathogens and to increase the total antibody recovery. Therefore, crude, pan-antibody purification methods are used for the purposes of the invention versus a more restrictive class-specific affinity purification method.

Physicochemical fractionation is an example of an extraction method that may be used to capture antibodies from breastmilk. This process involves separating antibodies using physical methods, chemical methods, electrical methods, or combinations thereof, into components, such as antibodies, from a sample. It may involve precipitation of antibodies (for example ammonium sulfate precipitation), size exclusion (for example dialysis membranes, size-exclusion resins, and diafiltration devices with high molecular weight cut-off), solid-phase binding (for example immobilized metal chelate chromatography), or separation by electrical charge (for example ion exchange chromatography). Ammonium sulfate precipitation, dialysis purification, and immunoglobulin column-binding purification may be used.

Class-specific affinity purification is another example of an extraction method that may be used to capture antibodies from breastmilk. Class-specific affinity purification may refer to solid-phase binding and/or biological ligands (for example jacalin, Protein A, Protein G, and Protein L) that capture all antibodies of a particular target class. The five primary immunoglobulin classes are IgA, IgD, IgE, IgG, and IgM, which are distinguished by their heavy chain.

Antigen-specific affinity purification is another example of an extraction method that may be used to capture antibodies from breastmilk. Antigen-specific affinity purification may refer to extraction of antibodies that only bind a particular antigen (without regard to antibody class or isotype). For example, the antigen of interest may be immobilized onto a solid support surface, a resin, or onto beads that enable purification and elution of corresponding antigen-specific antibodies.

Negative selection is another example of an extraction method that may be used to capture antibodies from breastmilk. Negative selection refers to the removal of unwanted components of breastmilk (for example albumin and casein). It may be desirable to remove certain components that do not contribute any beneficial nutritional and/or immune effect. In addition, some components may be removed if their presence may complicate efforts to stabilize and/or formulate the purified breastmilk antibodies of interest.

Various methods may be used to characterize antibodies derived from breastmilk. Each of these methods yields unique information about the structure or purity of the antibody sample. They may be used to ensure that the collected antibodies satisfy requirements for integrity (degradation) and contamination.

For example, mass spectrometry (MS) may be used for biophysical characterization of antibody preparations at the protein, peptide, and amino acid residue levels. This analytical equipment may be used to assess higher order structure, aggregation, and antibody complexation. For example, MS may be used to gauge whether antibodies are intact or have degraded into smaller peptides or amino acids.

As another example of a method to characterize antibodies, the yield and titer may be determined and analyzed. Yield and titer are related, but have important differences. The yield refers to the total antibody quantity in the final preparation, calculated as the antibody concentration multiplied by the volume; antibody concentration may be derived from optical measurements. However, concentration and yield do not account for the functional activity of the antibody molecules in this preparation. Functional activity, or titer, is a functional concentration or dilution-factor of an antibody solution against a particular antigen. An ELISA immunoassay-based dilution series is a common method by which titer may be determined.

As another example of a method to characterize antibodies, the assessment and containment of contamination may be measured and analyzed. Biologically-derived extracts such as antibodies are often tested for contamination by microbes including viruses, fungi, parasites, bacteria, and bacterial lipopolysaccharide (LPS), also known as endotoxin. Assays that demonstrate an endotoxin-decontamination benefit of the antibody purification protocol are preferred. The commercial-grade antibody purification protocol will follow current good manufacturing practice (CGMP) requirements, which serve as preventive measures and precautions to help protect product and prevent contamination. Beyond the protections afforded by CGMP practices, purification kits may also be used to, for example, remove LPS from the biologically-derived extracts described herein.

Furthermore, X-ray crystallography may be used to characterize the antibodies. Crystallography is a technique by which the 3-dimensional structure of a molecule can be assessed, and has been used historically to derive the structure of antibodies. It may be used to determine whether purified antibodies have retained their typical Y-shaped structure after the aforementioned purification steps.

In a present example, ammonium sulfate and column purification techniques were used to purify the antibodies. The human breastmilk sample was clarified by centrifugation at about 13,000 RPM for about 60 minutes to remove all fat from colostrum and milk. After clarification (removal of solid particulates such as lipids and casein), ammonium sulfate precipitation [ASP] was used for precipitation of antibodies. A range of about 40-45% ammonium sulfate has been described for precipitation of IgG from blood sera, but a wider range of ammonium sulfate concentrations was used for the purposes of this embodiment to identify the optimal condition for antibodies obtained from breastmilk as opposed to blood sera.

Following ASP, the samples were dialyzed in phosphate buffered saline (PBS) to remove ammonium sulfate and other residues, then further enriched for antibodies with an immunoglobulin [Ig] purification column containing pan-human capture antibodies for IgA, IgG and IgM bound to Sepharose 4B. Column elution buffer at pH 2.8, such as the standard for hybridoma-derived antibody elution, was compared against pH 4.0. The pH 2.8 buffer facilitated antibody capture shown by Western Blot, while pH 4.0 yielded no detectable antibodies in the eluant. To quantify the antibody samples that were detected by Western blot, optimal densitometry [OD] was used to measure protein concentration and obtained readings of 0.1 mg/ml, confirming that the ASP and Ig purification methods described herein yield human antibodies. Upon completion of the purification steps outlined herein, antibody samples were suspended in phosphate buffered saline (PBS), also referred to as “saline” or simply “buffer” by those skilled in the art.

In the present example, the efficiency of ammonium sulfate immunoglobulin precipitation increases consistently from about 30% to about 40% to about 45% to about 65% ammonium sulfate. The 65% ammonium percentage is much greater than the standard 40%, illustrating that the protocols derived for serum extractions are not sufficiently effective for breastmilk extractions.

It is to be appreciated by a person of skill that the ASP process alters protein solubility, driving aggregation, which helps precipitate out the protein, often referred to as “salting out” proteins from the solution. Given the size, susceptibility to aggregation, solubility, and surface charge of pathogens and other contaminants are different from antibodies, contaminants such as bacteria, viruses, and allergens would not be expected to co-precipitate with the antibody fraction. However, the ASP and Ig purification methods described above provide an additional and unexpected benefit of sample decontamination in this example.

In the present example, cellular neutralization assays were designed and executed to test the antiviral efficacy of human milk-derived polyclonal antibodies, referred to herein as LCTG-001, against RSV, as described below and in the Figures. Each of the following conditions was tested in quadruplicate wells: Cells alone; cells+GFP-RSV; cells+GFP-RSV preincubated with LCTG-001; cells+LCTG-001.

Cells: 5,000 HEp-2 (human airway epithelial) cells seeded per well in a 96-well plate; incubate for 12 hours.

LCTG-001: 10 micrograms of milk antibodies, corresponding to 90 microliters, were used per well.

Virus: GFP-RSV is an RSV strain engineered to express the fluorescent GFP protein. Cells infected with GFP-RSV emit fluorescence. RSV was added to HEp-2 cells at a multiplicity of infection (MOI) of 0.5, corresponding to approximately 2,500 plaque-forming units (PFU) of GFP-expressing RSV per well. Prior to being added to each well, RSV was preincubated with either LCTG-001 or sterile saline in a total volume of 100 microliters for 1 hour.

Cellular Incubation: HEp-2 cells were incubated with sterile saline, RSV, RSV+LCTG-001, or LCTG-001 alone, under standard cell culture conditions for 90 minutes to allow RSV infection. Supernatant was then aspirated, fresh cell culture medium was added, and cells were incubated for an additional 48 hours under standard cell culture conditions.

Quantification of Infection: After the 48 hour incubation, HEp-2 cells were stained with Hoechst 33342 (a commonly-used dye that binds double-stranded DNA in healthy cells) for 45 minutes and imaged at 37° C./4% CO2. Eight images at 10× magnification were acquired per well on the Cellomics ArrayScan VTI and subsequent analysis was performed with CellProfiler software. To quantify percent of cellular infection by RSV, cells were categorized as Hoechst-positive but GFP-negative (uninfected), or Hoechst-positive and GFP-positive (infected) to derive a percent of infected cells. Each well's average GFP signal was calculated from its eight corresponding images, and each experimental condition's average and standard deviation was then calculated from its corresponding four wells.

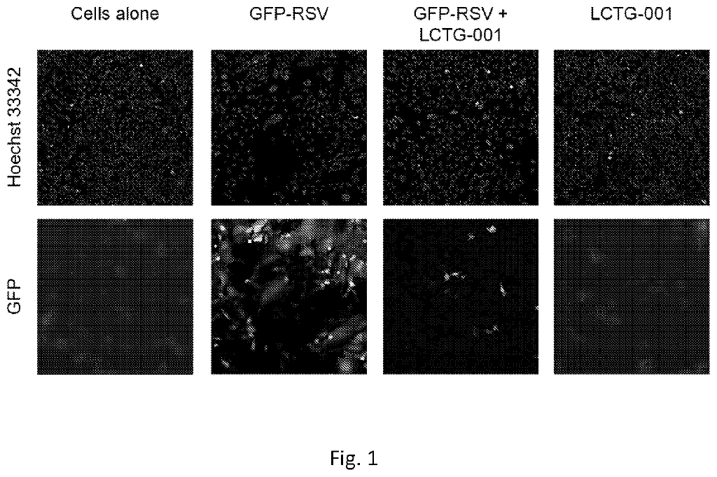

Referring to FIG. 1, upper row, the patterns of Hoechst-stained HEp-2 nuclei confirm that cell viability was qualitatively similar across all conditions, confirming that baseline cell health was maintained throughout all experimental conditions and establishing for the first time that LCTG-001 has no overt adverse effects on human airway cell viability. In the bottom row corresponding to GFP signal, uninfected cells displayed no signal while GFP-RSV-infected cells exhibited robust signal, as expected according to historical assay performance. Cells infected with GFP-RSV that had been preincubated with LCTG-001 exhibited very little GFP, indicating that the preincubation substantially reduced RSV infectivity. Lastly, cells incubated with LCTG-001 but no virus exhibited no GFP signal, as expected.

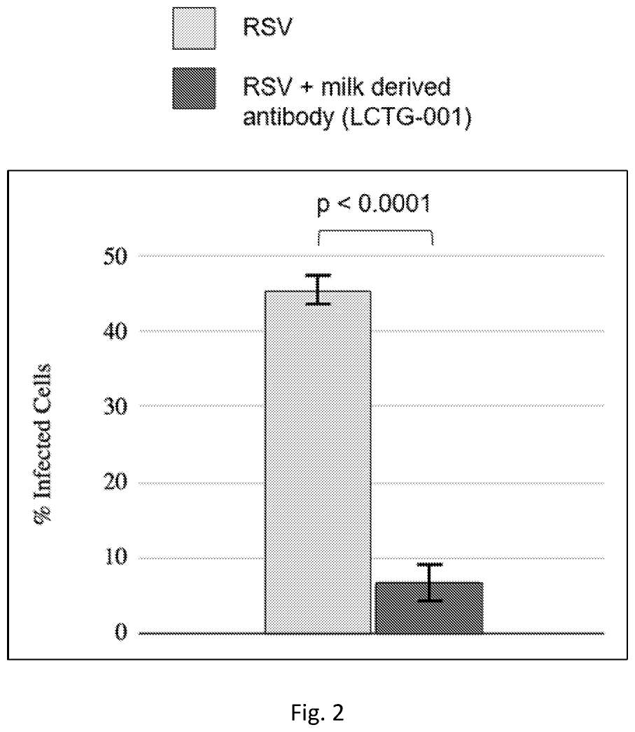

Referring to FIG. 2, GFP signals from the cells described in FIG. 1 were quantified and calculated as cellular infection averages and standard deviations in cells infected with GFP-RSV compared to cells infected with GFP-RSV preincubated with LCTG-001. In the absence of LCTG-001, RSV infected 45.54%±1.90 of cells, where 45.54 is the average percent of infected cells and 1.90 is the standard deviation. In the presence of LCTG-001, only 6.58%+2.42 of cells were infected, corresponding to an 85% reduction in RSV infection. It is noted that these experiments were conducted in HEp-2 epithelial cells in the absence of other immune factors or cells, indicating that milk antibodies possess inherent neutralizing properties. In this assay, LCTG-001 was stored in a freezer at −20° C. for one year with a single freeze-thaw cycle prior to performing the experiment.

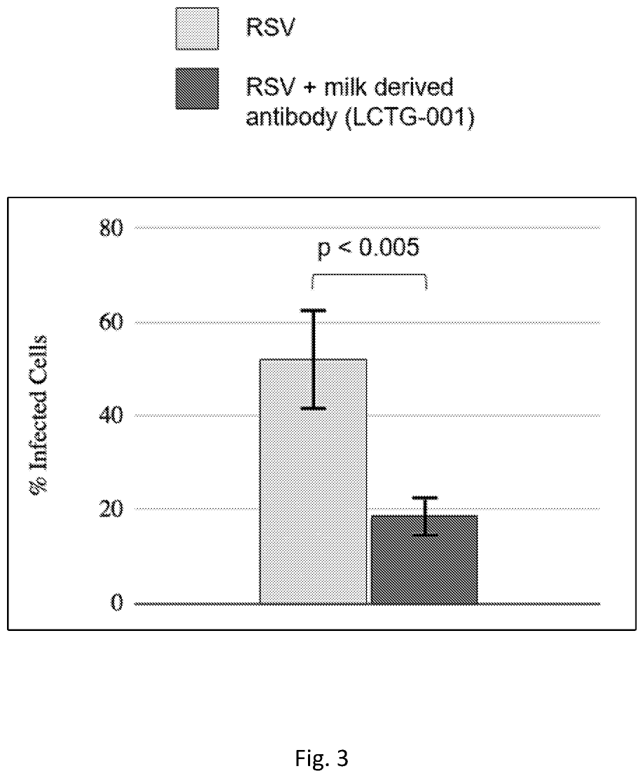

Referring to FIG. 3, LCTG-001 underwent extended storage at −20° C. for three years, with two freeze-thaw cycles (the first after one year as described in FIG. 2 and the second after three years). In the absence of LCTG-001, RSV infected 52.22%±10.48 of cells; of note, this is a similar percentage of infection as the study performed two years prior (specifically 45.54%±1.90), demonstrating the consistency of the cellular assay. In the presence of LCTG-001, only 18.65%±3.86 of cells were infected, corresponding to a 64% reduction in RSV infection. Accordingly, it is demonstrated that LCTG-001 may be stored at −20° C. for multiple years and still retain a majority of efficacy.

In summary, the ability of polyclonal human milk antibodies to neutralize RSV by 64-85% in vitro, in the absence of any other cellular or molecular components of human immunity, supports their utility as an RSV therapeutic for prophylactic and/or treatment purposes. The persistent bioactivity of human milk antibodies, despite 3 years of freezer storage, is a desirable and unexpected attribute that is not typically associated with biologic products.

It should be recognized that features and aspects of the various examples provided above may be combined into further examples that also fall within the scope of the present disclosure.

1. A composition for inhalation to treat respiratory syncytial virus, the composition comprising a polyclonal antibody derived from human breastmilk, wherein the composition comprises intact antibodies representing more than about 81% of total antibodies, the total antibodies comprising the intact antibodies and degraded antibodies.

2. The composition of claim 1, wherein the composition is stable for at least about 24 hours at about 25° C.

3. The composition of claim 2, wherein the composition is stable for at least about two weeks at about 25° C.

4. The composition of claim 1, wherein the composition is to be nebulized for delivery.

5. The composition of claim 1, wherein the composition is stable for at least about 1 year at about −20° C.

6. The composition of claim 5, wherein the composition is stable for at least about 3 years at about −20° C.

7. (canceled)

8. An apparatus for delivering a composition to treat respiratory syncytial virus, the apparatus comprising:
a storage reservoir to store the composition;
a nebulizer to provide a mist of the composition to be delivered to a dose of the composition; and
a delivery system to deliver the dose, wherein the composition comprises a polyclonal antibody mixture derived from human breastmilk.

9. A method of preparing a composition for inhalation to treat respiratory syncytial virus, the method comprising:
collecting a quantity of milk from a mammalian donor;
purifying a non-pasteurized sample of the quantity of milk to extract a polyclonal antibody fraction with ammonium sulfate precipitation with an ammonium sulfate concentration of about 45% to 65%;
characterizing the antibody fraction to determine the type of antibodies present;
providing a pooled composition of antibodies for use in the preparation of an inhalation dosage form.

10. The method of claim 9, wherein the antibody fraction is further characterized to determine the amount of antibodies present.

11. The method of claim 9 or 10, wherein the antibody fraction is nonspecific.

12. The method of claim 9, wherein the sample is purified using ammonium sulfate precipitation with an ammonium sulfate concentration of about 65%.

13. The method of claim 9, wherein the pooled composition of antibodies is at least about 90% IgA.
